(19) United States
(12) Patent Application Publication
(10) Pub. No.: US 2021/0268307 A1
(43) Pub. Date: Sep. 2, 2021

(54) PULSED LASER SYSTEM FOR DERMATOLOGICAL TREATMENTS

(71) Applicant: IRISIOME, Pessac (FR)

(72) Inventors: Romain ROYON, Dax (FR); Romain DUBRASQUET, Sabres (FR)

(21) Appl. No.: 17/276,088

(22) PCT Filed: Sep. 12, 2019
(86) PCT No.: PCT/EP2019/074342

§ 371 Date: Mar. 12, 2021

(30) Foreign Application Priority Data
Sep. 13, 2018 (FR) 1858247

(51) Int. Cl. A61N 5/06

(52) U.S. Cl. CPC A61N 5/0616; A61N 2005/067

(57) ABSTRACT

Disclosed is a pulsed laser system for dermatological treatment, including a light source suitable for emitting a light pulse beam and an optical amplifier system suitable for generating a laser pulse beam at a first repetition frequency. The duration of a laser pulse is between 100 femtoseconds and 100 picoseconds, the first repetition frequency is between 1 kHz and 10 GHz, each laser pulse having a quantity of energy less than or equal to 1 microjoule, and the pulsed laser system also includes a unit for temporally modulating the laser pulse beam and/or a unit for spatially modulating the laser pulse beam, the temporal and/or spatial modulation means being suitable for reducing the density of energy deposited on a surface to be treated and for generating a density of energy of between 0.0001 J/cm² and 0.01 J/cm².

### TECHNICAL FIELD TO WHICH THE INVENTION RELATES

The present invention generally relates to the field of laser systems.

It more particularly relates to a pulsed laser system for dermatological treatments applied to the human or animal body.

### TECHNOLOGICAL BACK-GROUND

The use of laser pulses finds many applications in the scientific, industrial or medical fields.

In dermatology, different lasers are used for many applications such as depilation, skin rejuvenation, pigmentary stain or tattoo removal.

Most currently available lasers, suitable for dermatological applications, generate long and energetic pulses that induce a thermal photo-ablation effect. In the present document, it is meant by “long pulses”, laser pulses of duration longer than several hundreds of picoseconds (ps), generally pulses of sub-nanosecond, nanosecond or microsecond duration. It is meant by “energetic pulse”, a laser pulse producing an energy at least of the order of a Joule. Lasers producing such long and energetic pulses operate at a low pulse-repetition frequency, generally lower than 10 hertz (Hz), for example between 1 Hz and 10 Hz.

In most known dermatological laser systems, the user, for example a dermatologist, holds a handpiece that allow him/her to apply the long, energetic and low repetition frequency laser pulse beam to an area of the skin to be treated. The laser beam is generally extended over a circular area having a diameter of several millimetres. When the laser beam covers the whole surface area to be treated, it is not necessary to move the laser beam. In certain cases, the user moves the handpiece step by step to treat another area of the skin. Moreover, the spot size limits the spatial resolution of these laser treatments.

However, the use of such a laser system is not without risk, in particular of local ablation and/or overheating liable to produce a burning sensation for the patient. Moreover, the efficiency of certain laser treatments in dermatology is unperfect, which requires several laser treatment sessions, for example for tattoo removal. Focusing the spot in a bid to increase the spatial resolution would entail an increase of the laser intensity, which would further increase the overheating risks.

Finally, considering the variety of treatments and types of skin, it is often necessary to have several specific laser systems, each system being dedicated to one application.

It is desirable to develop a laser system for dermatological application that is at the same time more efficient, less invasive, of better spatial resolution, while reducing the overheating or burning risks for the patient. It is desirable to develop a laser system for dermatological treatment application that is more compact and less expensive than the laser systems of the prior art.

### OBJECT OF THE INVENTION

In order to remedy the above-mentioned drawback of the state of the art, the present invention proposes a pulsed laser system for dermatological treatment, the pulsed laser system comprising a light source suitable for emitting a light pulse beam and an optical amplifier system suitable for receiving the light pulse beam and generating a laser pulse beam at a first repetition frequency.

More particularly, it is proposed according to the invention a pulsed laser system for dermatological treatment in which the duration (d) of a laser pulse is between 100 femtoseconds and 100 picoseconds, the first repetition frequency is between 1 kHz and 10 GHz, each laser pulse having a quantity of energy lower than or equal to 10 microjoule, and the pulsed laser system further includes means for temporally modulating the laser pulse beam and/or means for spatially modulating the laser pulse beam, these temporal and/or spatial modulation means being adapted to reduce the density of energy deposited on a surface area to be treated and to generate a density of energy between 0.0001 J/cm² and 0.01 J/cm².

According to a first embodiment, the means for temporally modulating the laser pulse beam comprise an acousto-optic modulator and an electric generator, the acousto-optic modulator being arranged at the output of the optical amplifier system, the electric generator being adapted to generate a radiofrequency signal applied to the electrodes of the acousto-optic modulator, the radiofrequency signal being adapted so that the acousto-optic modulator selects a burst of laser pulses.

According to a second embodiment, the optical amplifier system includes an optical pumping device and a current-voltage source and the means for temporally modulating the laser pulse beam comprise an electric generator adapted to generate a radiofrequency signal applied to the current-voltage source of the optical pumping device, the radiofrequency signal being adapted so that the optical amplifier system generates a burst of laser pulses.

Particularly advantageously, the electric generator is moreover adapted to temporally modulate the radiofrequency signal in such a way as to modulate in intensity the laser pulses of a burst of laser pulses.

Preferably, a burst of laser pulses includes a number N of laser pulses between 100 and 1,000,000.

According to a third embodiment, the means for spatially modulating the laser pulse beam comprise a beam scanning device, the beam scanning device being adapted to move the laser pulse beam on a region of interest of the surface area to be treated in such a way as to limit the density of energy deposited in the region of interest.

Particularly advantageously, the beam scanning device is adapted to move the laser beam along two transverse axes.

Preferably, the moving speed of the light beam in the region of interest is between 0.1 m/s and 10 m/s.

According to another particular and advantageous aspect, the laser system further includes an optical focusing system, the optical focusing system being adapted to focus the laser beam to a spot having a diameter lower than about 20 mm.

Other non-limitative and advantageous characteristics of the pulsed laser system for dermatological treatment according to the invention, taken individually or according to all the technically possible combinations, are the following:
the light source and the optical amplifier system are adapted to emit the laser pulse beam at a wavelength between 480 nm and 10,600 nm.

The invention also proposes a method for generating a pulsed laser beam for dermatological treatment, comprising the following steps:
emitting a light pulse beam and optically amplifying the light pulse beam to generate a laser pulse beam at a first repetition frequency, the duration (d) of a laser pulse being between 100 femtoseconds and 100 picoseconds, the first repetition frequency being between 1 kHz and 10 GHz, each laser pulse having a quantity of energy lower than or equal to 1 microjoule, and
temporally modulating the laser pulse beam and/or spatially modulating the laser pulse beam, in such a way as to reduce the density of energy deposited on a surface area to be treated and to generate a density of energy between 0.0001 J/cm² and 0.01 J/cm².

### DETAILED DESCRIPTION OF AN EXEMPLARY EMBODIMENT

The following description in relation with the appended drawings, given by way of non-limitative examples, will allow a good understanding of what the invention consists of and of how it can be implemented.

In the appended drawings:

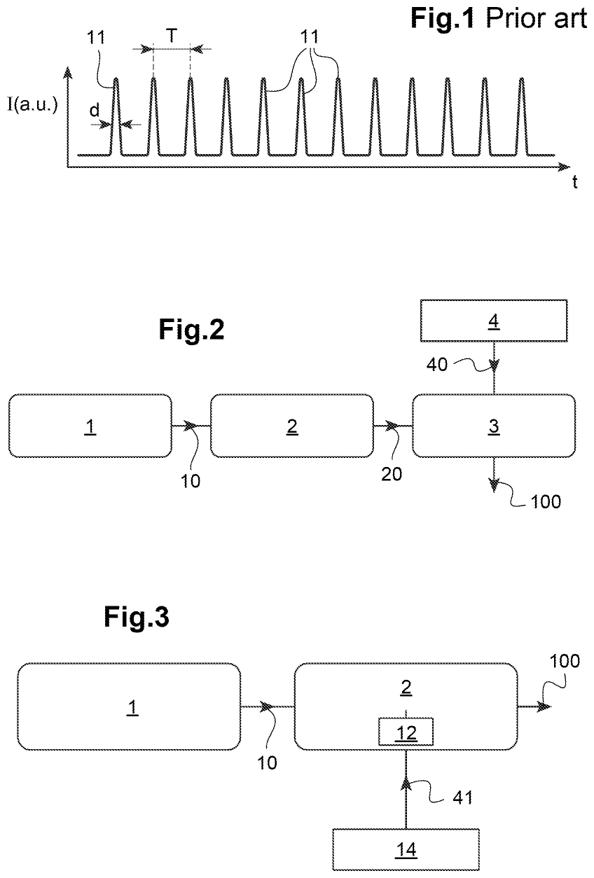

FIG. 1 schematically shows, as a function of time, the light intensity produced by a high-rate and long-pulse laser;

FIG. 2 schematically shows a laser system according to a first embodiment;

FIG. 3 schematically shows a laser system according to a second embodiment;

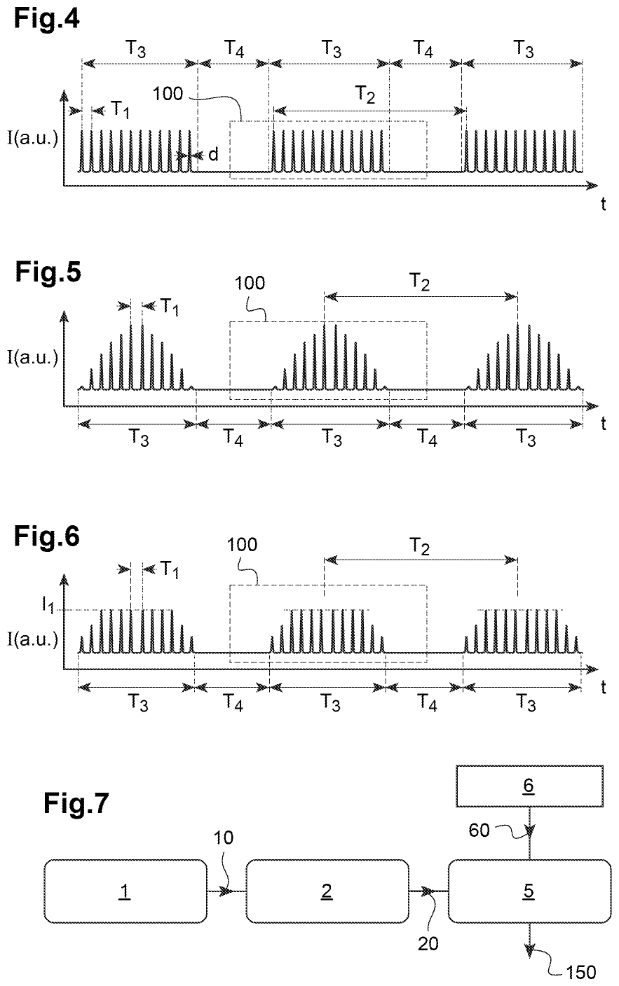

FIG. 4 schematically shows, as a function of time, a first example of light intensity produced by a laser according to the first or the second embodiment;

FIG. 5 schematically shows, as a function of time, a second example of light intensity produced by a laser according to the first of the second embodiment;

FIG. 6 schematically shows, as a function of time, a third example of light intensity produced by a laser according to the first of the second embodiment;

FIG. 7 schematically shows a laser system according to a third embodiment;

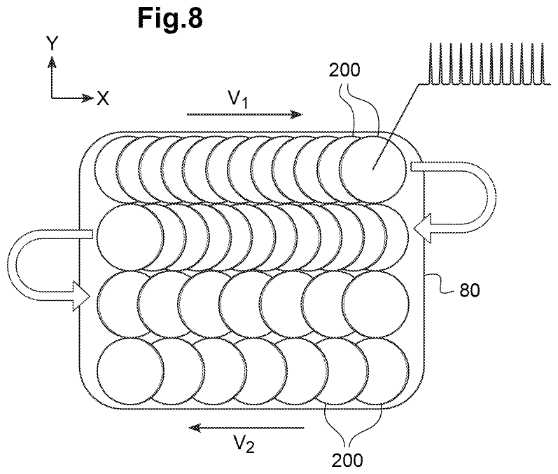

FIG. 8 schematically shows the operation of a laser system according to the third embodiment.

### DEVICE AND METHOD

In FIG. 1 is shown, as a function of time, the light intensity produced by a laser system generating long pulses according to the prior art. The duration of a laser pulse 11 is denoted d. For long pulses, this duration d is longer than 100 picoseconds (ps), generally of the nanosecond or microsecond order. The repetition period of the laser pulses 11 is denoted T. The repetition period T is equal to the inverse of the repetition frequency F. The repetition period T of the long laser pulses 11 is generally between 10⁻¹ s and 1 s. In other words, the repetition frequency F of the laser pulses 11 is between 1 Hz and 10 Hz. Such a laser system, which generates long laser pulses 11 at a repetition frequency F, produces an energy of the order of a millijoule or a joule per pulse. The laser pulse is incident on a generally circular extended area having a diameter of several millimetres on the surface area to be treated. In the conventional tattoo removal applications, the diameter of the laser beam is generally of the order of 5 mm. In the conventional depilation applications, the diameter of the laser beam is generally of the order of 18 mm.

Such a laser system generates a deposit of a quantity of energy per unit surface area, for a given time. This energy deposit is between 0.1 J/cm² and 100 J/cm², and generally of the order of 1 J/cm² per laser pulse having a pulse duration from ˜500 ps to several nanoseconds. The duration of treatment of an area to be treated is generally between 5 minutes and 60 minutes. Different physical phenomena may be involved in this energy deposit range. For laser pulses 11 having a duration longer than a nanosecond (10⁻⁹ s), it is considered that the absorption of the laser pulse beam is linear and induces a photo-ablation of the tissues. For laser pulses 11 having a duration shorter than a nanosecond (10⁻⁹ s) and a density of energy of the order of 1 J/cm², it is considered that the absorption of the laser pulse beam is non-linear and induces a photo-disruption of the tissues.

The present disclosure proposes different laser systems that use short or ultra-short laser pulses, having a duration d between about 100 femtoseconds and 100 picoseconds, and preferably shorter than 10 ps. According to the present disclosure, the laser system generates these short or ultra-short laser pulses at a first, high repetition frequency F₁, between 1 kHz and 10 GHz. In other words, the short or ultra-short laser pulses have a first repetition period T₁ between 100×10⁻¹⁵ s and 100×10⁻⁹ s. Each light pulse has a quantity of energy lower than or equal to 100 microjoules. Moreover, the laser system includes means for temporally modulating the laser beam and/or means for spatially modulating the laser beam, these temporal and/or spatial modulation means being adapted to reduce the density of energy deposited on the surface area to be treated.

Particularly advantageously, the laser system according to the present disclosure includes an optical system adapted to focus the laser beam to an area having a surface area of about 0.01 square millimetres, i.e. a surface area about 1000 times smaller than with a laser system for dermatological treatment according to the prior art.

FIG. 2 shows a pulsed laser system for dermatological treatment according to a first embodiment. The laser system is based on a laser diode, fibre laser, solid laser, dye laser or gas laser technology. The laser system includes an oscillator 1, an optical amplifier 2, an acousto-optic modulator 3 and an electric generator 4. The oscillator 1 generates a light pulse beam 10. The optical amplifier 2 receives the light pulse beam 10 and generates a laser pulse beam 20.

The laser pulse beam 20 has a wavelength generally between 700 nm and 10,600 nm. The duration d of the laser pulses 20 produced by the laser system is between 500 fs and 100 ps, and preferably shorter than 50 ps. The first repetition frequency F₁ of the laser pulses produced is between 1 kHz and 10 Ghz.

The acousto-optic modulator 3 is connected to the electric generator 4. More precisely, the electric generator 4 is adapted to generate a radiofrequency signal 40 applied to the electrodes of the acousto-optic modulator 3. The acousto-optic modulator 3 receives the laser pulse beam 20 and produces a temporally modulated laser pulse beam 100.

According to a first example of the first embodiment, the electric generator 4 is adapted to generate a door-shaped or rectangular radiofrequency signal 40, having a duration T₃ longer than the first repetition period T₁. The first repetition period T₁ is equal to the inverse of the first repetition frequency F₁: T₁=1/F₁. Hence, the acousto-optic modulator 3 selects a plurality of laser pulses forming a macro-pulse, also called pulse burst. Thus, the acousto-optic modulator blocks the laser pulses for a duration T₄ longer than the first repetition period T₁. This temporal modulation of the laser pulses makes it possible to limit the density of energy deposited on the surface area to be treated. Preferably, the so-deposited density of energy is lower than 0.01 J/cm² on a focused spot having a diameter between 10 μm and 20 mm, and preferably lower than ˜1 mm.

The duration T₃ of a macro-pulse is equal to the product of the number N of laser pulses in a macro-pulse by the first repetition period T₁ of the laser pulses. Each laser pulse of the macro-pulse has a quantity of energy lower than or equal to 1 microjoule.

Optionally, the acousto-optic modulator 3 is adapted to sequentially generate several macro-pulses. The acousto-optic modulator can be adapted to generate a plurality of macro-pulses with a second repetition period T₂. The second repetition period T₂ is equal to the sum of the duration T₃ of a rectangle and the duration T₄ between two consecutive rectangles. In other words, the radiofrequency signal 40 is zero for the duration T₄. The selection of duration T₃ and duration T₄ makes it possible to modulate the duration T₂ of a macro-pulse and the repetition frequency of the macro-pulses, herein denoted second repetition frequency F₂=1/T₂. That way, the acousto-optic modulator makes it possible to reduce the number of laser pulses incident on the surface area to be treated and to modulate the density of energy deposited.

FIG. 4 illustrates, as a function of time t, the light intensity of the temporally modulated laser pulse beam 100 generated by a laser system schematically illustrated in FIG. 2, in which the electric generator 4 applies a rectangular radiofrequency signal 40. In this example, the light pulses of a macro-pulse have all approximately the same light intensity.

According to a variant, the electric generator 4 produces a radiofrequency signal 40 that is modulated in intensity for a duration T₃ and zero for a duration T₄. More precisely, for a duration T₃, the radiofrequency signal 40 is increasing then decreasing. For example, the radiofrequency signal 40 has a triangular shape for the duration T₃. Optionally, the radiofrequency signal 40 is periodic with a second repetition period T₂ equal to the sum of duration T₃ and duration T₄. As illustrated for example in FIG. 5, such a radiofrequency signal 40 applied to the acousto-optic modulator 3 makes it possible to modulate the light intensity of the laser pulses incident on the surface area to be treated, by eliminating laser pulses not only outside the macro-pulse(s), for the duration T₄, but also inside each macro-pulse. A macro-pulse is hence obtained, containing N laser pulses, and in which the light intensity of the laser pulses is increasing then decreasing. As illustrated in FIG. 5, this macro-pulse can be repeated periodically at the second repetition period T₂, i.e. with the second repetition frequency F₂.

According to another alternative, the electric generator 4 produces a radiofrequency signal 40 that is modulated in intensity for a duration T₃ and zero for a duration T₄. More precisely, in this other alternative, for a duration T₃, the radiofrequency signal 40 is increasing then constant then decreasing. For example, the radiofrequency signal 40 has a trapezoidal shape for the duration T₃. Optionally, the radiofrequency signal 40 is periodic with a second repetition period T₂, equal to the sum of duration T₃ and duration T₄. As illustrated for example in FIG. 6, such a radiofrequency signal 40 applied to the acousto-optic modulator 3 makes it possible to modulate the light intensity of the laser pulses incident on the surface area to be treated, by eliminating laser pulses not only outside the macro-pulse(s), for the duration T₄, but also inside each macro-pulse. A macro-pulse is hence obtained, containing N laser pulses, and in which the light intensity of the laser pulses is increasing, constant over several successive pulses, then decreasing. As illustrated in FIG. 6, this macro-pulse can be repeated periodically at the second repetition period T₂, i.e. with the second repetition frequency F₂.

According to a second embodiment, the laser system has no acousto-optic modulator but contains an electric generator 14. The optical amplifier 2 includes an optical pumping device 12, for example one or several single-mode or multi-mode laser diodes, or also one or several flash lamps. The electric generator 4 is connected to the current-voltage source of the optical pump device. More precisely, the electric generator 14 is adapted to generate a radiofrequency signal 41 applied to the current-voltage source of the optical pumping device. The optical amplifier 2 receives the light pulse beam 10 from the oscillator and generates a temporally modulated laser pulse beam 150. Thus, the electric generator 14 makes it possible to directly modulate the light intensity of the pulses amplified by the optical amplifier 2.

According to a first example of this second embodiment, the electric generator 14 produces an intensity-modulated radiofrequency signal 41, for example of rectangular shape, non-zero for a duration T₃, the radiofrequency signal 41 being zero for a duration T₄. Thus, in this first example, for the duration T₃, the radiofrequency signal 41 is constant. Optionally, the radiofrequency signal 41 is periodic with a second repetition period T₂, equal to the sum of duration T₃ and duration T₄. As illustrated for example in FIG. 4, such a radiofrequency signal 41 applied to the current-voltage source of the optical pumping device 12 of the optical amplifier system 2 makes it possible to modulate the light intensity of the amplified laser pulses 100, by eliminating laser pulses outside the macro-pulse(s). A macro-pulse is hence obtained, containing N laser pulses, and in which the light intensity of the laser pulses is constant over the N successive pulses. As illustrated in FIG. 4, this macro-pulse can be repeated periodically at the second repetition period T₂, i.e. with the second repetition frequency F₂.

According to a first alternative of this second embodiment, the electric generator 14 produces an intensity-modulated radiofrequency signal 41, for example of triangular shape for a duration T₃, the radiofrequency signal 41 being zero for a duration T₄. Thus, in this alternative, for the duration T₃, the radiofrequency signal 41 is increasing then decreasing. Optionally, the radiofrequency signal 41 is periodic with a second repetition period T₂. As illustrated for example in FIG. 5, such a radiofrequency signal 41 applied to the current-voltage source of the optical pumping device of the optical amplifier system makes it possible to modulate the light intensity of the amplified laser pulses inside a macro-pulse. A macro-pulse is hence obtained, containing N laser pulses, and in which the light intensity of the laser pulses is increasing then decreasing. As illustrated in FIG. 5, this macro-pulse can be repeated periodically at the second repetition period T₂, i.e. with the second repetition frequency F₂.

According to another alternative of this second embodiment, the electric generator 14 produces an intensity-modulated radiofrequency signal 41, for example of trapezoidal shape for a duration T₃, the radiofrequency signal 41 being zero for a duration T₄. Thus, in this alternative, for the duration T₃, the radiofrequency signal 41 is increasing then decreasing. Optionally, the radiofrequency signal 41 is periodic with a second repetition period T₂. As illustrated for example in FIG. 5, such a radiofrequency signal 41 applied to the current-voltage source of the optical pumping device of the optical amplifier system makes it possible to modulate the light intensity of the amplified laser pulses inside a macro-pulse. A macro-pulse is hence obtained, containing N laser pulses, and in which the light intensity of the laser pulses is increasing, constant over several successive pulses, then decreasing. As illustrated in FIG. 6, this macro-pulse can be repeated periodically at the second repetition period T₂, i.e. with the second repetition frequency F₂.

The person skilled in the art will easily adapt the shape and durations T₃ and T₄ of the radiofrequency signal 40 or 41 to obtain the light intensity modulation of the laser pulses as a function of the application and the dermatological treatment considered.

This second embodiment makes it possible to modulate the pump signal of the optical amplifier to select a plurality of laser pulses forming a macro-pulse, in such a way as to limit the density of energy deposited by the laser beam to less than 0.01 J/cm².

Due to the limited energy of the laser pulse beam, the present disclosure makes it possible to focus the laser beam on a smaller surface area. The spot is hence 1000 times smaller than a laser spot generated by a dermatological laser system of the prior art. Instead of covering a surface area of a few square millimetres, the laser beam then covers only a surface area of about 0.01 square millimetres, i.e. a laser beam focused on a disk having a diameter of about 50 to 100 micrometres. However, certain dermatological treatments must be performed on large surface area, of several square centimetres to a few tens of square centimetres. The use of a laser spot having a diameter lower than about a tenth of mm can take a significant amount of time, which is a priori dissuasive.

However, according to the present disclosure, a first repetition frequency F₁ is used, which is 10,000 to 100,000 times higher than the repetition frequency F of a laser system of the prior art. The use of a laser system of very high repetition frequency F₁ makes it possible to obtain a cumulative effect on the treated surface area and to induce a non-linear photo-disruptive effect. In other words, the laser system of the present disclosure operates in a new regime. Indeed, in this new regime, each individual pulse has not the sufficient energy or light intensity to produce optical ruptures of the photo-disruptive type. However, thanks to the non-linear processes, the cumulative interactions of a plurality of laser pulses at the first repetition frequency F₁ induce structural changes in the materials, entailing an improvement of the absorption. After a determined number of pulses, which depends on the surface area to be treated and on a light intensity threshold, a photo-disruption phenomenon occurs. The major interest of this new effect is linked to the fact that the density of energy deposited on the skin is limited and preferably lower than or equal to 0.01 J/cm², which makes it possible to preserve the surface area treated in dermatology from undesirable phenomena of ablation and overheating.

The cumulative interaction of a series of N microjoule-energy laser pulses at a first high repetition frequency F₁ makes it possible to obtain a selective photo-disruptive effect as a function of the concerned target, non-limitatively chosen, for example, among endogenous pigments, exogenous pigments, melanin or sebum.

According to a third embodiment, which can be implemented separately from or in combination with the first or the second embodiment, the laser system includes an oscillator 1, an optical amplifier system 2, and further includes a beam scanning device 5, also called a scanner. An electric control and synchronization system 6 is connected to the beam scanning device 5. The beam scanning device 5 is arranged at the output of the laser chain, herein at the output of the optical amplifier system 2. By way of non-limitative example, the beam scanning device 5 includes a scanner with two transverse axes. Such a beam scanning device 5 makes it possible to move the laser beam along two transverse directions on the surface area to be treated. The electric control and synchronization system 6 generates an electric or electronic signal 60 that makes it possible to control the moving orientation, direction and speed on each axis of the beam scanning device 5.

The electric control and synchronization system 6 and the beam scanning device 5 are configured to move the laser beam on the surface area to be treated in such a way as to limit the density of energy deposited, preferably to less than 0.01 J/cm². As a function of the moving speed, it is thus possible to modify the quantity of energy deposited per unit surface area for a given time, i.e. to modulate the density of energy deposited on the treated surface area.

FIG. 8 illustrates an example of operation of the pulsed laser system combined to a beam scanning device 5. FIG. 8 shows a surface area to be treated 80. An XY reference system is shown in the plane of the surface area to be treated 80. This surface area to be treated 80 extends for example over a square of 1 mm side. An optical system focuses the laser pulse beam of low energy per pulse and high repetition frequency F₁ to form a spot 200 in the surface area to be treated 80. The spot 200 is generally circular in shape and has a diameter of about 500 μm. The optical focusing system can be arranged upstream or downstream from the beam scanning device 5.

The treatment is started, for example, by applying a laser spot 200 on the top left of the surface area to be treated 80. The beam scanning device 5 moves the laser beam along the axis X with a first moving speed V1. Arrived near the edge of the surface area to be treated 80, the beam scanning device 5 applies a move along axis Y and changes the direction along axis X while keeping the first moving speed V1. Arrived near the other edge of the surface area to be treated 80, the beam scanning device 5 applies a move along axis Y and changed the direction along axis X, while applying a second moving speed V2 higher than V1. Arrived near the edge of the surface area to be treated 80, the beam scanning device 5 applies a move along axis Y and changes the direction along axis X, while keeping the second moving speed V2. The moving speeds may reach 8 m/s, which makes it possible to cover the surface area to be treated 80 of 1 cm² for a spot of 1 mm within ˜13 ms.

The moving speed determines the number of cumulated high-rate pulses and hence makes it possible to determine the quantity of energy deposited per unit surface area for a given time. Hence, the density of energy deposited is higher in the area in which the beam is moved at speed V1, by comparison with the area of the surface area to be treated in which the beam is moved at speed V2.

The moving speed along axis X or Y is generally between 0.1 m/s and 10 m/s, and preferably higher than 5 m/s.

The laser beam may be moved continuous or step by step.

The third embodiment makes it possible to spatially modulate the density of energy of a low-energy laser pulse beam having a high repetition period F₁, so as to strongly reduce the density of energy deposited. This third embodiment hence makes it possible to reach the cumulative photo-disruption non-linear interaction regime.

According to a particular embodiment, the laser system combines a temporal modulation of the laser pulses into macro-pulses, as described in relation with the first or the second embodiment, and a laser spot move on the area to be treated as described in relation with the third embodiment. This combination of a temporal and spatial modulation of the laser pulse beam makes it possible to increase the dynamics of adjustment of the density of energy that is deposited. It also makes it possible to improve the spatial resolution of the laser beam without increasing the total duration of the laser treatment for a given surface area.

A single laser system combining an acousto-optic modulator and a beam scanning device 5 makes it possible to modulate both temporally and spatially the density of energy of the high repetition frequency and low energy laser pulses over a very wide dynamics.

Still more simply, a single laser system combining an optical amplifier system and a beam scanning device 5, in which an electric RF signal generator is connected to the current-voltage source of the optical pump device of the optical amplifier system makes it possible to modulate both temporally and spatially the density of energy of the high repetition frequency and low energy laser pulses over a very wide dynamics.

A single laser system combining the first and the third embodiments or the second and third embodiments hence makes it possible to perform a varied range of dermatological treatments, which previously required several laser systems.

The laser system according to the present disclosure operates in a new interaction regime, herein called cumulative photo-disruption of the tissues, which is based on the use of a number N of laser pulses, preferably ultra-short, of low energy, high repetition frequency, and on a temporal and/or spatial modulation of the laser beam to limit the density of energy deposited on the surface area to be treated.

The invention makes it possible to focus the beam on spot of very small size, which allows a better targeting of the area to be treated. The spatial covering of the area to be treated is obtained by moving the laser beam, preferably by means of a beam scanning device. This beam scanning can be adjusted as a function of the contours of the surface area so as to apply the laser beam on the whole surface area to be treated 80 without going over the lines about this surface area to be treated.

1. A pulsed laser system for dermatological treatment, the pulsed laser system comprising a light source suitable for emitting a light pulse laser and an optical amplifier system suitable for receiving the light pulse beam and generating a laser pulse beam at a first repetition frequency (F₁), wherein:
the duration (d) of a laser pulse is between 100 femtoseconds and 100 picoseconds, the first repetition frequency (F₁) is between 1 kHz and 10 GHz, each laser pulse having a quantity of energy lower than or equal to 1 microjoule, and the pulsed laser system further includes means for temporally modulating the laser pulse beam and/or means for spatially modulating the laser pulse beam, these temporal and/or spatial modulation means being adapted to reduce the density of energy deposited on a surface area to be treated and to generate a density of energy between 0.0001 J/cm² and 0.01 J/cm².

2. The system according to claim 1, wherein the means for temporally modulating the laser pulse beam comprise an acousto-optic modulator and an electric generator, the acousto-optic modulator being arranged at the output of the optical amplifier system, the electric generator being adapted to generate a radiofrequency signal applied to the electrodes of the acousto-optic modulator, the radiofrequency signal being adapted so that the acousto-optic modulator selects a burst of laser pulses.

3. The system according to claim 1, wherein the optical amplifier system includes an optical pumping device and a current-voltage source, and wherein the means for temporally modulating the laser pulse beam comprise an electric generator adapted to generate a radiofrequency signal applied to the current-voltage source of the optical pumping device, the radiofrequency signal being adapted so that the optical amplifier system generates a burst of laser pulses.

4. The system according to claim 2, wherein the electric generator is further adapted to temporally modulate the radiofrequency signal in such a way as to modulate in intensity the laser pulses of a burst of laser pulses.

5. The system according to claim 2, wherein a burst of laser pulses includes a number (N) of laser pulses between 100 and 1,000,000.

6. The system according to claim 1, wherein the means for spatially modulating the laser pulse beam comprise a beam scanning device, the beam scanning device being adapted to move the laser pulse beam on a region of interest of the surface area to be treated in such a way as to limit the density of energy deposited in the region of interest.

7. The system according to claim 6, wherein the beam scanning device is adapted to move the laser beam along two transverse axes.

8. The system according to claim 6, wherein the moving speed of the light beam in the region of interest is between 0.1 m/s and 10 m/s.

9. The system according to claim 1, further comprising an optical focusing system, the optical focusing system being adapted to focus the laser beam to a spot having a diameter lower than about 20 mm.

10. The system according to claim 1, wherein the light source and the optical amplifier system are adapted to emit the laser pulse beam at a wavelength between 700 nm and 10,600 nm.

11. The system according to claim 3, wherein the electric generator is further adapted to temporally modulate the radiofrequency signal in such a way as to modulate in intensity the laser pulses of a burst of laser pulses.

12. The system according to claim 3, wherein a burst of laser pulses includes a number (N) of laser pulses between 100 and 1,000,000.

13. The system according to claim 4, wherein a burst of laser pulses includes a number (N) of laser pulses between 100 and 1,000,000.

14. The system according to claim 2, wherein the means for spatially modulating the laser pulse beam comprise a beam scanning device, the beam scanning device being adapted to move the laser pulse beam on a region of interest of the surface area to be treated in such a way as to limit the density of energy deposited in the region of interest.

15. The system according to claim 3, wherein the means for spatially modulating the laser pulse beam comprise a beam scanning device, the beam scanning device being adapted to move the laser pulse beam on a region of interest of the surface area to be treated in such a way as to limit the density of energy deposited in the region of interest.

16. The system according to claim 4, wherein the means for spatially modulating the laser pulse beam comprise a beam scanning device, the beam scanning device being adapted to move the laser pulse beam on a region of interest of the surface area to be treated in such a way as to limit the density of energy deposited in the region of interest.

17. The system according to claim 5, wherein the means for spatially modulating the laser pulse beam comprise a beam scanning device, the beam scanning device being adapted to move the laser pulse beam on a region of interest of the surface area to be treated in such a way as to limit the density of energy deposited in the region of interest.

18. The system according to claim 7, wherein the moving speed of the light beam in the region of interest is between 0.1 m/s and 10 m/s.

19. The system according to claim 3, further comprising an optical focusing system, the optical focusing system being adapted to focus the laser beam to a spot having a diameter lower than about 20 mm.

20. The system according to claim 3, wherein the light source and the optical amplifier system are adapted to emit the laser pulse beam at a wavelength between 700 nm and 10,600 nm.
